(19) United States
(12) Patent Application Publication
(10) Pub. No.: US 2013/0052140 A1
(43) Pub. Date: Feb. 28, 2013

(54) FE3O4/TIO2 COMPOSITE NANO-PARTICLE, ITS PREPARATION AND APPLICATION IN MAGNETIC RESONANCE IMAGING CONTRAST AGENTS

(71) Applicants: Leyong Zeng, Ningbo (CN); Aiguo Wu, Ningbo (CN); Ping Cui, Ningbo (CN)

(72) Inventors: Leyong Zeng, Ningbo (CN); Aiguo Wu, Ningbo (CN); Ping Cui, Ningbo (CN)

(21) Appl. No.: 13/521,198

(22) PCT Filed: Oct. 15, 2010
(86) PCT No.: PCT/CN10/77803

§ 371 Date: Jul. 9, 2012

(30) Foreign Application Priority Data
Jan. 12, 2010 (CN) 201010039632.0

(51) Int. Cl. A61K 49/06; B82Y 40/00; B82Y 5/00; B82Y 30/00; B82Y 25/00

(52) U.S. Cl. 424/9.32; 977/773; 977/811; 977/838; 977/930; 977/840

(57) ABSTRACT

This invention provides a Fe₃O₄/TiO₂ composite nano-particle, its preparation and application in the magnetic resonance imaging (MRI) contrast agent, wherein the preparation of Fe₃O₄/TiO₂ composite nano-particles has the followings steps: trivalent iron compounds and bivalent iron compounds are dissolved into a reducing acid water solution, and then added with tetravalent titanium salt solution to obtain the Fe₃O₄/TiO₂ composite nano-particle sol. The Fe₃O₄/TiO₂ composite nano-particles prepared by the method in the invention have the properties of superparamagnetism and photocatalytic activity and can be applied to MRI contrast agents.

### FIELD OF THE INVENTION

The present invention relates to nano-particles, preparation and application thereof, especially Fe₃O₄/TiO₂ composite nano-particles, their preparation and application in Magnetic Resonance Imaging contrast agents.

### BACKGROUND OF THE INVENTION

At present, Magnetic Resonance Imaging (MRI) is one of the most important imaging technologies in clinic diagnosis of any part of human body in modern medicine because of its advantages of safety, high imaging resolution, less artifacts and abundant information. In medical diagnosis, the MRI sensitivity bears a very important relationship with the contrast agent materials used. The material that can be used as an MRI contrast agent includes nano-materials such as super paramagnetic oxide nano-particles and gadolinium containing coordination compounds. Due to non-toxicity and relatively strong magnetic characteristics, the superparamagnetic iron oxide (Fe₃O₄) nano-particles are widely researched in the field of MRI T2 and/or T2* contrast agent materials, compared to the frequently used T1 contrast agent that is made of gadolinium coordination compounds. At present, the Fe₃O₄ nano-materials are often prepared by the coprecipitation method: ferric salt is firstly dissolved in a solvent and then vigorously stirred to obtain Fe₃O₄ nano-particles under basic conditions; and the products is naturally precipitated or magnetically separated to obtain Fe₃O₄ nano-particles. In this method, both inorganic/organic ferric salt and inorganic/organic solvents are used. Although Fe₃O₄ nano-materials can be made according to this method, the particle diameter is relatively large. In addition, the Fe₃O₄ nano-particles prepared by this method have poor water-soluability and poor bio-compatability, which limit their applications in biomedicine field, especially application in MRI contrast agents. Therefore, the problems of toxicity, solubility and biocompatibility shall be solved before the magnetic nano-materials are applied in MRI contrast agents and medical therapy.

In addition, individual Fe₃O₄ nano-particles only have T2 and/or T2* weighted signals with common signal intensity contrast in MRI tests. T1 weighted signals only occur if the particle size is very small. Moreover, superparamagnetic Fe₃O₄ nano-particle contrast agent is usually applied to medical imaging diagnosis, but very difficult to be combined with biological molecules or drug molecules in medical treatment.

Nano titanium dioxide (TiO₂) is an important photocatalytic material, widely used in the fields of chemical catalysis, energy and photodegradation of environmental pollutants. Using the photocatalytic activity of TiO₂ or linking TiO₂ nano-particles to biological molecules or drug molecules to form nano-copolymers, one can use the TiO₂ nano-materials in disease treatment.

Therefore, application of above said environmental-friendly nontoxic Fe₃O₄/TiO₂ composite nano-particles with good solubility and biocompatibility to the MRI contrast agent field helps develop a high-resolution and informative MRI contrast agent having T1, T2 and/or T2* weighted signals. The Fe₃O₄/TiO₂ composite nano-particles can have medical therapeutic effects if linked to drug molecules. It has great significance of the new MRI contrast agent made of Fe₃O₄/TiO₂ composite nano-particles in improving MRI test sensitivity, decreasing cost of medical test and therapy, and safeguarding people's life and health.

### SUMMARY OF THE INVENTION TECHNICAL PROBLEM

The technique issue involved in the present invention is how to apply Fe₃O₄/TiO₂ composite nano-particles of small particle size with high solubility and biocompatibility to preparation of MRI contrast agents.

### THE TECHNICAL SOLUTION

The invention provides a method of preparing Fe₃O₄/TiO₂ composite nano-particles to solve the issue above, comprising the following steps: trivalent iron compounds and bivalent iron compounds are dissolved into a reducing acid solution; and the solution is added with a tetravalent titanium salt solution to obtain the Fe₃O₄/TiO₂ composite nano-particle sol.

Optionally, the method further comprises the step that surfactants are added into the Fe₃O₄/TiO₂ composite nano-particle sol to regulate the nano-particle size, preventing nano-particles from aggregation and over growth. The preferred surfactant is cetyl trimethyl ammonium bromide or sodium dodecyl sulfate.

Optionally, the method further comprises the step that the obtained Fe₃O₄/TiO₂ composite nano-particle sol is subject to aging treatment in water bath at 20° C.-99 ° C. for over 20 hours to obtain nano-particles of even particle size which are consistent in the need of MRI contrast agents.

Optionally, the mole ratio of trivalent iron compound to bivalent iron compound is from 2:1 to 1:1, and the mole ratio of bivalent iron compound to reducing acid is from 1:12 to 1:1. Through creative work, the inventors found the materials of these ratios can obtain small Fe₃O₄/TiO₂ composite nano-particles of even size.

Optionally, the reaction is at 0° C.-100° C. Optionally, the concentration of the tetravalent titanium salt solution is from 0.01 mol/L to 5 mol/L. The inventors found the tetravalent titanium salt solution of this concentration at this temperature can prepare Fe₃O₄/TiO₂ composite nano-particles of a controllable particle size.

In addition, another aspect of the invention relates to the Fe₃O₄/TiO₂ composite nano-particles prepared by the method.

Optionally, TiO₂ accounts for 10%-90% of nano-particles in weight. The inventors found that TiO₂ of this weight percent can ensure the Fe₃O₄/TiO₂ composite nano-particles have better magnetism and MRI signals, and maintain the biocompatibility of TiO₂ components.

Another aspect of the invention is that the Fe₃O₄/TiO₂ composite nano-particles are applied to MRI contrast agents.

### Beneficial Results

In the present invention, the method of preparing Fe₃O₄/TiO₂ composite nano-particles has the advantages of simple operation, low cost, evenly distributed and controllable particle size, non-toxicity, good solubility and biocompatibility without the need for N₂ protection during the reaction process.

The Fe₃O₄/TiO₂ composite nano-particles prepared by the method in the invention is a composite nano material having both super paramagnetism and photocatalytic activity, which has wide application prospects in medical imaging diagnosis and medical treatment. Super paramagnetic Fe₃O₄ components can have medical imaging function and separate the photo-generated holes on the TiO₂ surface from ions, which prolongs the bonding time of holes and ions, improves the photocatalytic activity and enhance the therapeutic effects of TiO₂ components. Moreover, TiO₂ components can protect Fe₃O₄ components from oxidation. The MRI contrast agent made of Fe₃O₄/TiO₂ composite nano-particles can have T1, T2 and/or T2* weighted signals and realize both medical diagnosis and therapeutic effects if TiO₂ components with good biocompatibility are linked to drug molecules by covalent bonds to form nano copolymers.

### BRIEF DESCRIPTION OF THE DRAWINGS

FIG. 1 is the TEM diagram of Fe₃O₄/TiO₂ composite nano-particles prepared by an embodiment of the invention.

FIG. 2 is the HRTEM diagram of Fe₃O₄/TiO₂ composite nano-particles shown in FIG. 1.

FIG. 3 is the XRD diagram of Fe₃O₄/TiO₂ composite nano-particles prepared by an embodiment of the invention.

FIG. 4 is the magnetization curve of Fe₃O₄/TiO₂ composite nano-particles prepared by an embodiment of the invention.

FIG. 5 is a T1 and T2 weighted signal chart in MRI test of Fe₃O₄/TiO₂ composite nano-particles prepared by an embodiment of the invention.

### DESCRIPTION OF THE PREFERRED EMBODIMENT

The Fe₃O₄/TiO₂ composite nano-particles related to the invention are prepared by the following steps:

1. A certain amount of reducing acid is weighed and dissolved in water, and then stirred adequately to obtain the reducing acid solution with the concentration of 0.01 mol/L to 1 mol/L;

2. Trivalent ferric salt and bivalent ferric salt with the ratio of 2:1 to 1:1, among which the ratio of Fe²⁺ and reducing acid is 1:12 to 1:1, are added into the reducing acid solution, and then stirred adequately at 0° C.-99° C., preferably 20° C.-30° C. for less than an hour;

3. The ferric salt solution is put into ice bath and added with the tetravalent titanium salt solution of 0.01 mol/L-5 mol/L molar concentration in a dropwise manner until the TiO₂ component accounts for 10%-90% of the total TiO₂ and Fe₃O₄ components, and then stirred adequately for over one hour;

5. The solution is added with appropriate surfactants after reaction and continues to be stirred for over 30 min;

6. The prepared solution is subject to aging treatment in water bath at 20° C. above for more than 20 hours to obtain the Fe₃O₄/TiO₂ composite nano-particle sol;

7. The obtained Fe₃O₄/TiO₂ composite nano-particle sol is packed into reagent bottles and put into a refrigerator at 4° C. for MRI contrast agent; or the sol is dialyzed, frozen and dried to obtain a powder product for MRI contrast agents.

The surfactant is cetyl trimethyl ammonium bromide (CTAB) or sodium dodecyl sulfate (SDS), which can effectively improve nano-particles aggregation phenomenon.

The Fe₃O₄/TiO₂ composite nano-particles obtained by above method can be evenly dispersed in water and water solution to form transparent lyosol, which proves that the sol has a good dispersibility. Moreover, the sol can be kept at 4° C. for more than 1 year, which shows the Fe₃O₄/TiO₂ composite nano-particle sol have good solubility and stability.

In addition, the Fe₃O₄/TiO₂ composite nano-particles also have very good biocompatibility as Fe₃O₄ and TiO₂ have very good biocompatibility.

The above implementation method is described in detail with the following embodiments.

### EMBODIMENT 1

(1) Citric acid of 3 mmol was taken to dissolve in deionized water of 350 mL, and vigorously stirred for full dissolution of citric acid to obtain the citric acid solution;

(2) FeCl₃.6H₂O of 2 mmol and FeCl₂.4H₂O of 1 mmol were taken to dissolve in the citric acid solution, and adequately stirred;

(3) The reaction vessel was placed in the ice bath and stirred for 30 min.

(4) The reaction vessel was added with 0.1 mol/L TiCl₄ solution of 10 mL in a dropwise manner and continued to be stirred;

(5) After 5 hours, the product was put into a water bath boil at 65° C. and subject to aging treatment for 22 hours;

(6) The product was dialyzed, frozen and dried to obtain Fe₃O₄/TiO₂ composite nano-particle powder.

### EMBODIMENT 2

(1) Citric acid of 6 mmol was taken to dissolve in deionized water of 350 mL, and vigorously stirred for full dissolution of citric acid to obtain the citric acid solution;

(2) FeCl₃.6H₂O of 4 mmol and FeCl₂.4H₂O of 2 mmol were taken to dissolve in the citric acid solution, and adequately stirred;

(3) The reaction vessel was placed in the ice bath and stirred for 30 min.

(4) The reaction vessel was added with 0.6 mol/L TiCl₄ solution of 1 mL in a dropwise manner and continued to be stirred;

(5) After 5 hours, the product was added with 0.8 mmol/L CTAB of 50 mL and stirred for 30 min;

(6) The product was put into a water bath boil at 65° C. and subject to aging treatment for 20 hours.

(7) The product was dialyzed, frozen and dried to obtain Fe₃O₄/TiO₂ composite nano-particle powder.

### EMBODIMENT 3

(1) Citric acid of 6 mmol was taken to dissolve in deionized water of 350 mL, and vigorously stirred for full dissolution of citric acid to obtain the citric acid solution;

(2) FeCl₃.6H₂O of 2 mmol and FeCl₂.4H₂O of 2 mmol were respectively taken to dissolve in the said citric acid solution, and adequately stirred;

(3) The reaction vessel was placed in the ice bath and stirred for 30 min.

(4) The reaction vessel was added with 0.1 mol/L TiCl₄ solution of 20 mL in a dropwise manner and continued to be stirred;

(5) After 5 hours, the product was added with 0.8 mmol/L SDS of 50 mL and stirred for 30 min;

(6) The product was put into a water bath boil at 65° C. and subject to aging treatment for 20 hours.

(7) The product was dialyzed, frozen and dried to obtain Fe₃O₄/TiO₂ composite nano-particle powder.

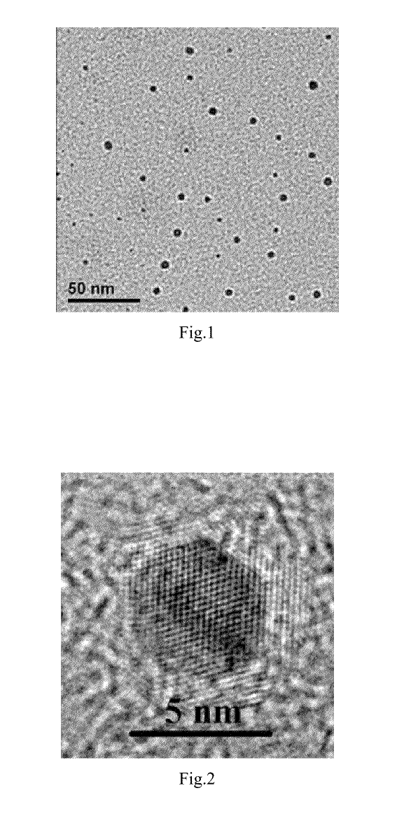

FIG. 1 and FIG. 2 are TEM and HRTEM diagrams of Fe₃O₄/TiO₂ composite nano-particles prepared by said Embodiment 1. The Fe₃O₄/TiO₂ composite nano-particles have good dispersibility and even particle size (average size: about 5 nm for a single particle) as shown in FIG. 1 and FIG. 2.

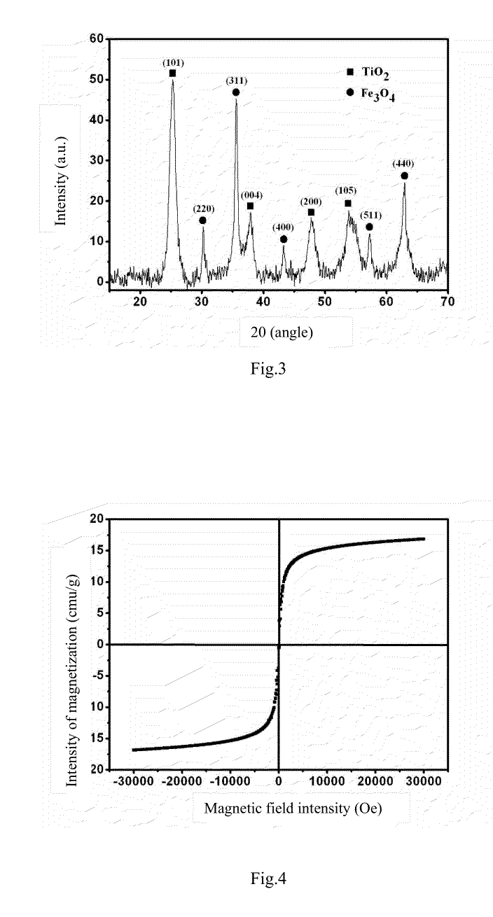

FIG. 3 is the XRD diagram of Fe₃O₄/TiO₂ composite nano-particles prepared by said Embodiment 1 (Cu Kα target, λ=0.15418 nm). Tetragonal TiO₂ and cubic Fe₃O₄ diffraction peaks as shown in FIG. 3 indicate that Fe₃O₄/TiO₂ composite nano-particles have the crystal structure of Fe₃O₄ and TiO₂.

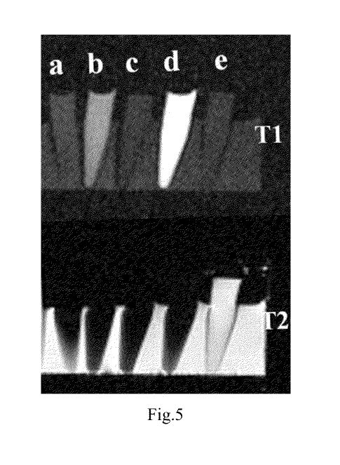

FIG. 4 is the magnetization curve of the Fe₃O₄/TiO₂ composite nano-particles prepared by said Embodiment 1, which was obtained with American Quantum Design comprehensive physical property test (Model-9, test conditions: VSM (vibrating sample magnetometer) test, temperature: 300 K). FIG. 4 shows the Fe₃O₄/TiO₂ composite nano-particles have the saturation intensity of about 16.95 emu/g, the coercive force and remanence of 0, indicating that Fe₃O₄/TiO₂ composite nano-particles have super paramagnetism. Therefore, Fe₃O₄/TiO₂ composite nano-particles can be effectively applied in MRI contrast agents.

FIG. 5 shows T1 and T2 weighted signal diagram in the MRI test of Fe₃O₄/TiO₂ composite nano-particles prepared by Embodiment 3. MIR test conditions are: TR=300 ms, TE=6 ms, T2:TR=4000 ms, TE=120 ms. Sample a, b and c are nano Fe₃O₄; sample d is the Fe₃O₄/TiO₂ composite nano-particle prepared by Embodiment 3; and e is the water solution for control.

FIG. 5 shows that all nano Fe₃O₄ samples have T2 weighted signals, but only the nano Fe₃O₄ sample b has relatively weak weighted signals T1. The Fe₃O₄/TiO₂ composite nano-particle sample prepared by Embodiment 3 has both strong T1 and T2 weighted signals.

The invention is disclosed as mentioned above, but it does not limit the claims. Any technician in this field can change and revise it without departing from the scope and essence of the invention. Therefore, the invention protects these defined in the Claims.

1. A method of preparing Fe₃O₄/TiO₂ composite nano-particles, wherein it comprises the following steps: trivalent iron compounds and bivalent iron compounds are dissolved into a reducing acid solution; and the solution is added with tetravalent titanium salt solution in a dropwise manner to obtain the Fe₃O₄/TiO₂ composite nano-particle sol.

2. The method of preparing Fe₃O₄/TiO₂ composite nano-particles according to claim 1, wherein it further comprises the step that a surfactant is added to the Fe₃O₄/TiO₂ composite nano-particles.

3. The method of preparing Fe₃O₄/TiO₂ composite nano-particles according to claim 2, wherein the surfactant is cetyl trimethyl ammonium bromide or sodium dodecyl sulfate.

4. The method of preparing Fe₃O₄/TiO₂ composite nano-particles according to claim 2, wherein the obtained Fe₃O₄/TiO₂ composite nano-particle sol is subject to aging treatment in water bath at 20° C.-99° C. for over 20 hours.

5. The method of preparing Fe₃O₄/TiO₂ composite nano-particles according to claim 1, wherein the mole ratio of trivalent iron compound to bivalent iron compound is from 2:1 to 1:1, and the mole ratio of bivalent iron compound to reducing acid is from 1:12 to 1:1.

6. The method of preparing Fe₃O₄/TiO₂ composite nano-particles according to claim 1, wherein the reaction temperature is at 0° C.-100° C.

7. The method of preparing Fe₃O₄/TiO₂ composite nano-particles according to claim 1, wherein the concentration of tetravalent titanium salt solution ranges from 0.01 mol/L to 5 mol/L.

8. Fe₃O₄/TiO₂ composite nano-particles obtained according to claim 1.

9. The Fe₃O₄/TiO₂ composite nano-particles according to claim 8, wherein TiO₂ accounts for 10% to 90% of nano-particles in weight.

10. The Fe₃O₄/TiO₂ composite nano-particles according to claim 8 are applied in MRI contrast agents.
